(19) United States
(12) Patent Application Publication
(10) Pub. No.: US 2005/0214870 A1
(43) Pub. Date: Sep. 29, 2005

(54) Assay

(75) Inventors: Pamela M. Greengrass, Sandwich (GB); Michael Stewart, Sandwich (GB); Claire Margaret Wood, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/795,187

(22) Filed: Mar. 4, 2004

(30) Foreign Application Priority Data
Sep. 4, 2001 (GB) 0121440.2
Sep. 4, 2002 (WO) PCT/IB02/03618

(51) Int. Cl. G01N 33/53; G01N 33/567

(52) U.S. Cl. 435/7.2

(57) ABSTRACT

The present invention relates to assays useful for predicting whether a compound has the effect to prolong the QT interval as measured by an electrocardiogram in a human. These assays make use of the binding action of dofetilide to the HERG K⁺ channel, and the propensity of a test compound to influence that binding action.

### FIELD OF THE INVENTION

The invention relates to an assay to establish the affinity of compounds at the human HERG K⁺ channel, using [³H]-dofetilide. This assay is useful to identify compounds with undesirable effects on cardiac repolarisation in man, in particular the propensity to prolong the QT interval in the electrocardiogram.

### BACKGROUND

In recent years the development of compounds has been aborted in late phase drug development due to undesirable effects on cardiac repolarisation in man. The effects of these drugs are assessed in terms of the QT interval in the electrocardiogram (ECG). The QT interval is the portion of an ECG that represents the time from the beginning of ventricular depolarization to the end of ventricular repolarization. Because the QT interval can be affected by heart rate lengthening with a decrease in heart rate and shortening with an increase in heart rate, the QT is often “corrected” for heart rate, resulting in the QT_{c} interval. In rare cases the application of some drug molecules results in a prolongation of the QT interval of the ECG in man. The ECGs of these patients resemble those of individuals suffering from an inherited disorder known as long QT syndrome. Drug-induced ventricular fibrillation, in these cases, can eventually lead to sudden death (Morganroth J et al. (1993) Am J Cardiol. 72, 26B-31B; De Ponti F. et al., (2000) Eur J. Clin. Pharmacol. 56, 1-18). A number of drug molecules, including, E-4031, cisapride and terfenadine, are all known to prolong the QT interval of the electrocardiogram in man (Fuliki A, et al. (1994), Cardiovascular Pharmacol. 23: 374-378; Van Haarst A D et al., (1998) Clin Pharmacol. Ther. 64: 542-546; Honig P. K. et al. (1993) J.A.M.A. 269; 1513-1518).

The launch of new drugs with undetected cardiotoxic side effects could have hazardous consequences and could trigger lethal cardiac dysrhythmias in patients. Late detection of QT prolongation, induced by compounds of pharmacological interest therefore can dramatically impede drug discovery and development programmes, and consequently have a profound impact on the financial outcome of a program. It is desirable, therefore, to test for the potential cardiotoxic side effects of compounds at an early stage of drug development. The invention provides a high throughput test system that provides a preclinical predictive indicator for drugs with the propensity to prolong the QT interval in man. Furthermore, in conjunction with structure-activity relationships (SAR), [³H]-dofetilide ligand binding assays can be used as a tool to assist the design of new drugs devoid of or with reduced affinity to HERG

Dofetilide is a selective inhibitor of the rapid component of the delayed rectifier potassium current which prolongs the action potential duration and the effective refractory period in a concentration-dependent manner. Clinical studies have demonstrated that dofetilide is effective in treating patients with atrial as well as ventricular arrhythmias. Dofetilide has formula I below.

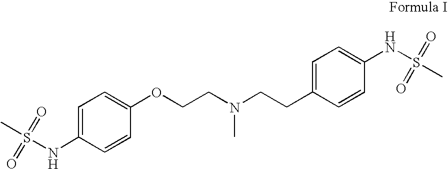

Dofetilide is claimed and its preparation is described in European patent EP 0245997.

### ASPECTS OF THE INVENTION

The main aspect of the invention is an assay useful for predicting whether a compound has the effect to prolong the QT interval in the electrocardiogram in man. In a preferred embodiment, the assay is a binding assay to the HERG K⁺ channel (ether-a-go-go K⁺ channel, herein called HERG), preferably human HERG, using labelled dofetilide.

In a preferred embodiment of the invention, the ligand used in the binding assay is labelled dofetilide, preferably radiolabeled dofetilide, most preferred [³H]-dofetilide.

The ligand binding assay of the invention preferably includes the following steps:
a) incubation of cells or membranes expressing the HERG K⁺ channel with labelled dofetilide in the presence or absence of different amounts of a test compound or a mixture of test compounds;
b) determination of specifically bound label for each sample;
c) calculation of the inhibition of labelled dofetilide binding by the test compound or mixture of test compounds.
   The assay may also include the step of calculating the concentration of compound that gives 20% inhibition of dofetilide binding (IC₂₀).

Preferred assay formats include the filter binding technique, whereby bound and unbound labelled dofetilide are separated by filtration, or the scintillation proximity assay technique well known to the person skilled in the art, using radiolabelled dofetilide.

### EXAMPLES

### LIST OF FIGURES

FIG. 1: Saturation curve data for [³H]-dofetilide binding to HERG in filter binding and SPA formats;

FIG. 2: Correlation plots comparing pKi values obtained from filter binding and SPA binding assays;

FIG. 3: Comparison of inhibition of [³H]-dofetilide binding to HERG, HERG patch clamp, and free drug concentration known to induce QT interval prolongation in man, for E-4031, dofetilide, terfenadine, and cisapride.

### EXAMPLE 1

### Preparation of Membranes from HEK293 Cells Expressing Human HERG

The adherent HEK293 cell line expressing human HERG (Zhou, Z et al (1998) Biophys. J. 74, 230-241) was provided under a licensing agreement by Dr. Craig January, University of Wisconsin, USA.

Adherent HEK293 cells expressing human HERG, were grown in MEM Earles medium (Life Technologies) supplemented with 10% foetal calf serum (PAA Laboratories), 2 mM L-glutamine (Sigma), 1 mM sodium pyruvate (Sigma), 0.4 mg/ml G418 (Life Technologies) and an addition of 1× non-essential amino acids (Life Technologies). The cells were grown at 37° C. in a humidified atmosphere with 5% CO₂ in T225 cm³ flasks. The cells were split 1:3-1:5 after reaching 80% confluence using cell dissociation solution (Sigma, cat no: C5914 in 2001) and later seeded into 850 cm² CO₂ gassed roller bottles (Corning, cat no: 430849 in 2001) in the absence of G418.

For the preparation of membranes, cells were harvested from the roller bottles by scraping and resuspended in PBS (Life Technologies, cat no: 14190-094 in 2001). All cells were pelleted, washed twice with PBS and snap-frozen on dry ice prior to storage at −80° C. until required.

Cell membrane fractions were prepared from frozen aliquots of cells. All procedures were carried out at 4° C. unless otherwise stated. Frozen aliquots of cells were thawed at room temperature and resuspended in homogenisation buffer (50 mM Tris base, 10 mM KCl, 1-1.2 mM MgCl₂, pH 7.4). The cells were then disrupted by homogenisation in an Omni LabTek homogeniser at 20,000 rpm for 30 seconds. The homogenate was centrifuged for 20 minutes at 48,000×g (4° C., Sorvall RC5B centrifuge) and the supernatant removed. The resulting pellets were resuspended in homogenisation buffer and homogenised as above for 10 seconds. The pellets were collected by centrifugation and the final pellet resuspended in buffer. Protein content was determined using a Coomassie Blue based protein assay kit. Aliquots were stored at −80° C. until needed. Binding proved to be stable for at least 4 months under the storage conditions mentioned above.

### EXAMPLE 2

### Filter Binding Assay with [³H]-dofetilide

[³H]-dofetilide (80-83 Ci/mmol) was synthesized by catalytic tritiation (a custom service provided, for example, by Amersham Life Science). However, other detectable labels known to the skilled person can be used instead of ³H, e.g. fluorescent tags, other radiolabels, etc.

On the day of the assay compounds were dissolved at 1 mM in 50% DMSO, and then diluted to the desired concentrations in binding buffer.

Incubations included membrane homogenate at 50 μg/ml in assay buffer (50 mM Tris base, 10 mM KCl, 1-1.2 mM MgCl₂, pH 7.4) unless otherwise indicated, [³H]-dofetilide (4-7 nM) and control vehicle or compounds to be assayed where appropriate. Filtration assays were incubated at room temperature for 90 minutes. Non-specific binding was determined in the presence of 10 μM dofetilide and was usually <15% of total binding. Bound ligand was separated from free ligand by rapid filtration through GF/B glass fibre filter mats using a Brandel cell harvester or onto GF/B Unifilter 96-well filter plates (Packard) using a Packard Filtermate 96 harvester. Filter mats and plates were pre-soaked in 5% PEI (w/v) for 60 minutes and washed after harvesting with 3×1 ml washes of ice-cold assay buffer. Unifilter plates were air dried for a minimum of 1.5 hours at 37° C. prior to the addition of Microscint-0 (Packard). Bound [³H]-dofetilide was determined by liquid scintillation spectroscopy in a Packard TopCount Scintillation Counter for Unifilter plates and in a Wallac Big Spot Counter when filter mats were used.

In each experiment, triplicate assays were routinely performed and the data were averaged. Specific binding was analysed by nonliiear regression fit using GraphPad Prism software (GraphPad, San Diego). IC₅₀ values were derived from a 4 parameter logistic fit using PRISM and converted to Ki values by use of the Cheng & Prusoff equation; IC₂₀ values were extrapolated from the graph.

### EXAMPLE 3

### Scintillation Proximity Assay

The scintillation proximity assay (SPA) was carried out under identical buffer conditions to those used in the filtration assay. Conditions were optimised with respect to bead and cell membrane homogenate concentration, prior to characterising HERG pharmacology. The incubations (200 μl total for 96 well plates and 60 μl total for 384 well plates) included 25 μg of cell membrane homogenate per mg of bead. The membrane homogenate was precoupled with the Yttrium silicate Wheatgerm Agglutinin bead suspension at 4° C. on a roller shaker for approximately 2 hours. For competition binding assays, membrane homogenate bead suspension was incubated in white clear bottom 96 or 384 well plates with 5 nM [³H]-dofetilide in the absence and presence of competitor compound. The plates were incubated at room temperature and shaken for approximately 1 hour. Beads were allowed to settle for a minimum of 30 minutes before plates were counted for retained radioactivity on a TopCount NTX scintillation counter. Nonspecific binding was determined in the presence of 10 μM dofetilide and was usually <15% of the total binding. For saturation studies, specific binding of [³H]-dofetilide was determined over a range of concentrations (5-500 nM) in the absence or presence of cold 10 μM dofetilide.

### EXAMPLE 4

### Assay Optimisation

a) Effect of Hepes- and Tris-Based Buffers on Dofetilide Binding

To optimise the specific binding of dofetilide to cell membrane homogenates containing HERG, the interaction of [³H]-dofetilide with this preparation was examined in the presence of Hepes-based buffer (25 mM Hepes, 135 mM NaCl, 5 mM KCl 1 mM MgSO₄, 50 mM CaCl₂, pH 7.4) and Tris-based buffer (50 mM Tris, 10 mM KCl, 1 mM MgCl₂). Comparison of the specific binding in these buffers revealed that % specific binding was similar in both Tris-based and Hepes-based buffers. However, specific counts doubled in the presence of Tris-based buffer (Table 1).

TABLE 1
Comparative effects of Tris based and Hepes based buffers on [³H]-dofetilide
binding to cell membrane homogenate expressing HERG.

| | 25 mM HEPES free acid | 50 mM TRIS |
|---|---|---|
| | 135 mM NaCl,5 mM KCI | 10 mM KCI |
| | 1 mM MgSO₄, 50 μM CaCI₂ | 1.2 mM MgCI₂ |
| BUFFER | (pH 7.4 at room temp) | (pH 7.4 at room temp) |
| TOTAL BINDING | 8510 ± 669 | 19627 ± 1189 |
| (ccpm) | | |
| NON SPECIFIC | 321 ± 27 | 315 ± 23 |
| BINDING (ccpm) | | |
| SPECIFIC BINDING | 8189 | 19312 |
| (ccpm) | | |
| % SPECIFIC BINDING | 96 | 98 |

Total and non specific binding data represents arithmetic mean±s.e.mean of 14 individual wells per buffer split over two assays. Performed at a protein concentration of 75 μg/ml and a mean [³H]-dofetilide concentration of 6.7 nM. Incubation was 60 minutes at room temperature. Ccpm=corrected counts per minute.

From this point on, all binding assays were carried out in the presence of Tris-based incubation buffer (50 mM Tris, 10 mM KCl, 1 mM MgCl₂) so that the maximum specific binding window was achieved. Additionally, experiments were performed to optimise the cell membrane protein concentration and bead concentration for filter and SPA binding assays (data not shown).

b) Saturation Binding

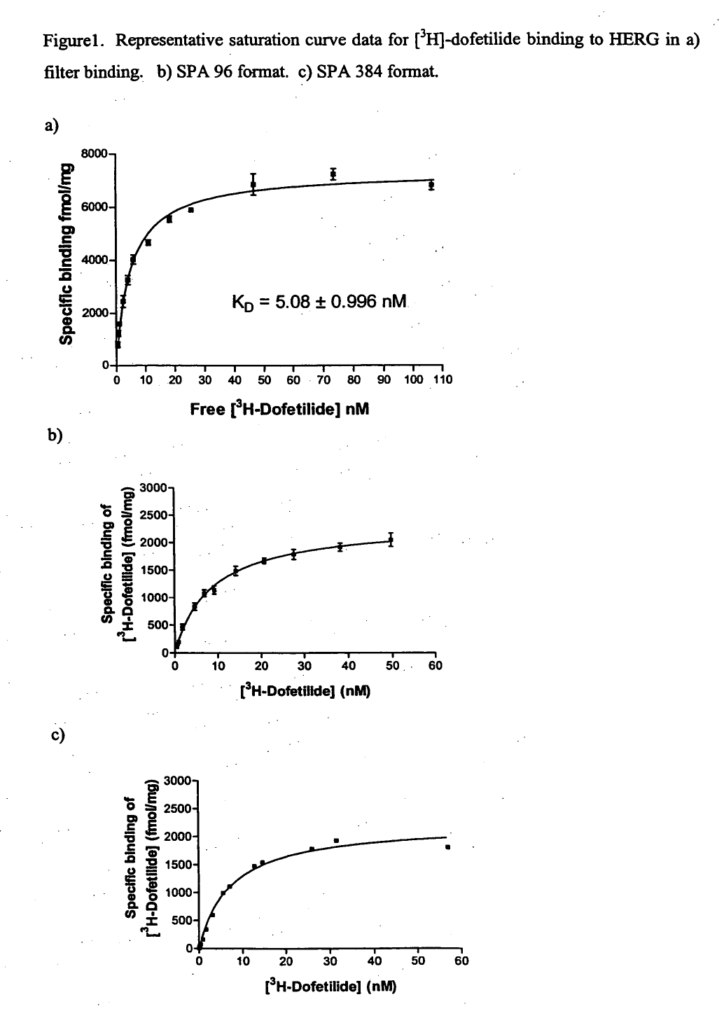

Time courses were performed to determine optimal incubation time for binding activities (data not shown). Incubation times were similar for both filter binding and SPA assays. The filter binding assay reached equilibrium in 90 minutes and SPA required 60 minutes. [³H]-dofetilide binding to HERG in both filter binding and scintillation proximity assays was saturable with a K_{D} of 5.08±1.0 nM for filter binding and K_{D} values of 8.9±0.5 nM and 9.1±1.8 nM for 96 and 384 format scintillation proximity assays respectively (FIG. 1a-c, with FIG. 1a showing the results of the filter binding assay, FIG. 1b the results of the SPA in 96-well format, and FIG. 1c showing the results of the SPA in 384 well format). Non-linear curve fitting of this data indicated that binding was to a single site. A Bₘₐₓ of 7.4±0.7 pmol/mg (FIG. 1) protein for [³H]-dofetilide was obtained from filter binding. As scintillation proximity assays do not give an accurate determination of dpm (disintegrations per minute) values, a Bₘₐₓ is not quoted for SPA.

c) Comparison of SPA and Filter Binding Techniques

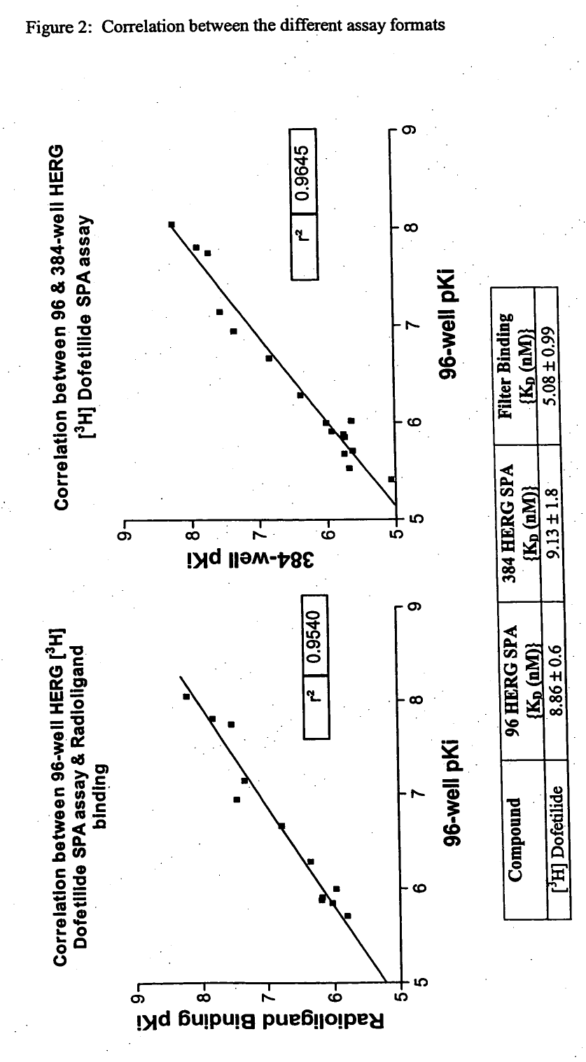

A comparison of SPA and filter binding techniques reveals excellent concordance of results. Affinity values display excellent correlation between the two assay types and the rank order of compound affinity is identical, as is shown in FIG. 2 (correlation plots comparing pKi values obtained from filter binding and SPA binding assays).

d) Competitive Binding Studies

A range of compounds, including HERG blockers known to prolong the QT interval in man, were examined for competitive displacement of [³H]-dofetilide. E4031, dofetilide, terfenadine, and cisapride produced complete inhibition of specific binding with a range of calculated affinity values that are summarised in Table 2.

TABLE 2
Affinity values for compounds tested against [³H]-dofetilide
filter and SPA binding assays to HERG.

| | Filter | | |
|---|---|---|---|
| | binding | SPA 96 | SPA 384 |
| Compound | pKᵢ | pKᵢ | pKᵢ |
| Dofetilide | 8.22 ± 0.04 | 8.05 ± 0.54 | 8.26 ± 0.12 |
| E4031 | 7.82 ± 0.03 | 7.81 ± 0.05 | 7.89 ± 0.11 |
| Terfenadine | 7.53 ± 0.09 | 7.75 ± 0.07 | 7.72 ± 0.41 |
| Cisapride | 7.34 ± 0.05 | 7.15 ± 0.04 | 7.55 ± 0.22 |
| Glibenclamide | <5 | <5 | <5 |
| D-Sotalol | <5 | <5 | <5 |

Data expressed as pKi values (the negative logarithm of molar concentration of competing ligand to displace 50% of 5 nM [³H]-dofetilide binding) Data are the mean of at least n = 3 experiments

### EXAMPLE 5

### Prediction of QT Interval Prolongation Effect of Compounds in Man

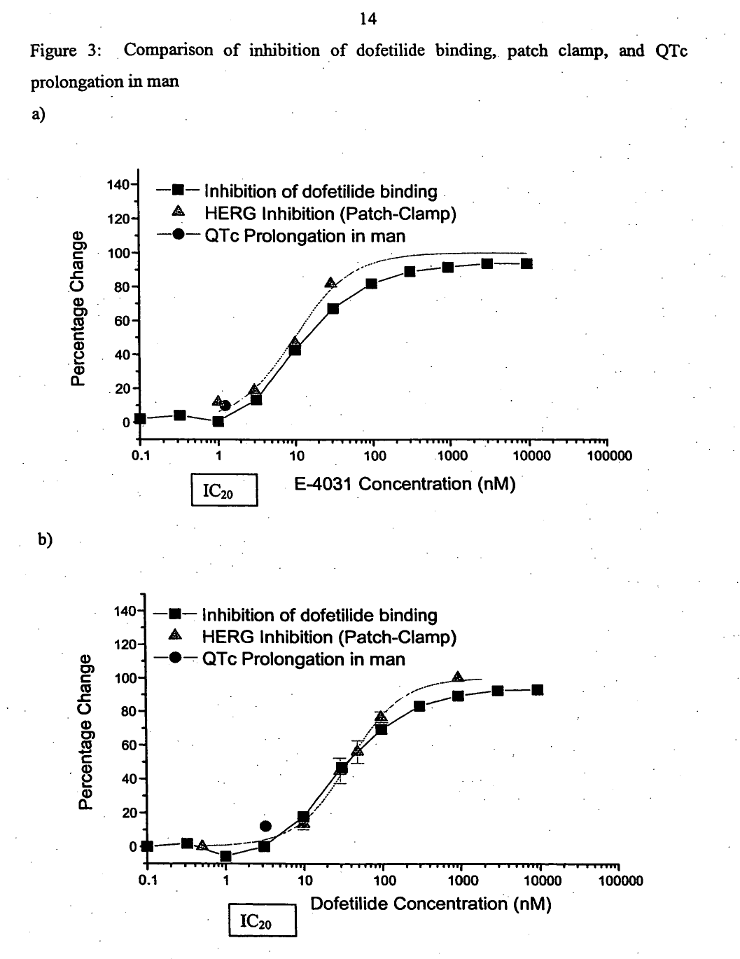

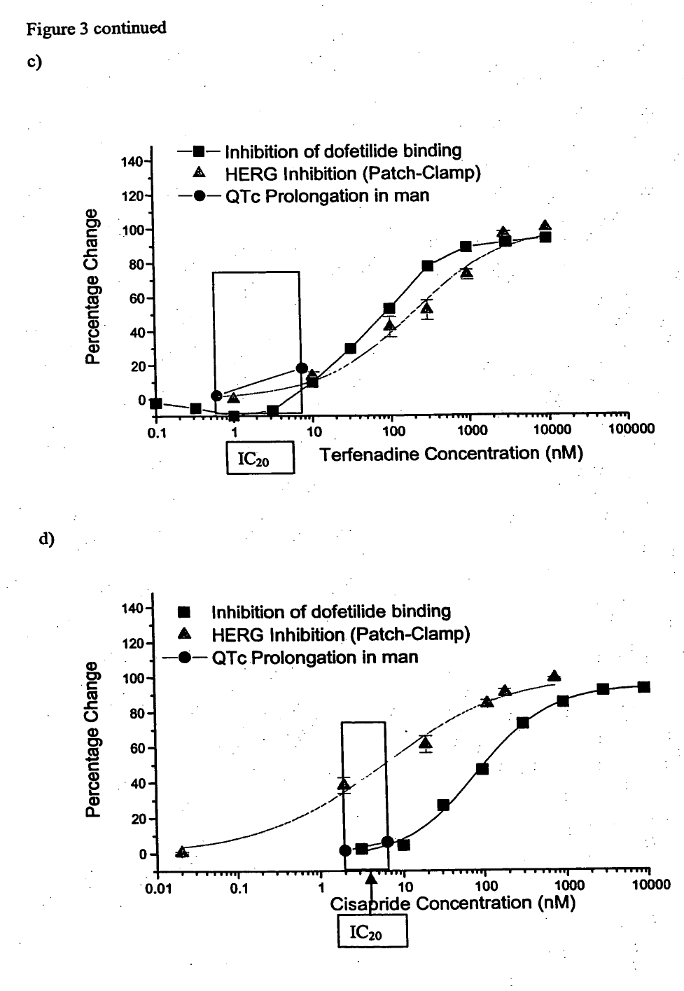

The IC₂₀ values generated from competitive displacement of [³H]-dofetilide binding are comparable to the free drug concentration associated with QT prolongation in man as is shown in FIG. 3 for a range of compounds, including E-4031 (FIG. 3a), dofetilide (FIG. 3b), terfenadine (FIG. 3c) and cisapride (FIG. 3d). For each compound, the inhibition of dofetilide binding in the binding assay (filter binding technique), and in a HERG patch clamp assay is compared with the concentration of free drug associated with QT interval prolongation in man (Fuliki A, et al. (1994) Cardiovascular Pharmacol, 23: 374-378; Van Haarst A D et al. (1998) Clin Pharmacol. Ther. 64: 542-546; Honig P K, et al. (1993) J.A.M.A. 269: 1513-1518).

Due to the phenomena of state dependent block observed in patch clamp studies (Walker, B. D. et al (1999) British J. Pharmacol 128, 444-450) exhibited by a number of known HERG blockers with the propensity to prolong the QT interval in vivo, ligand binding is a better predictor of in vivo QT prolongation of drug molecules than HERG patch clamp (FIG. 3d).

Therefore, to assess whether a compound is likely to prolong the QT interval in the electrocardiogram in man, the following steps are carried out:
a) A binding assay is carried out essentially as described in Example 2 or Example 3, to test the affinity of the compound to the dofetilide binding site on HERG;
b) The IC₂₀ is obtained as described at the end of Example 2;
c) The IC₂₀ is the real or predicted free drug concentration at which QT prolongation occurs in man;
d) The IC₂₀ value is therefore compared with the free drug concentration required for the desired therapeutic effect of the compound.
e) If the free drug concentration required for the desired therapeutic effect of the compound is within 10-30 fold of the IC₂₀ of the compound in the dofetilide binding assay, the compound is likely to show QT interval prolongation in man.

1. An assay useful for predicting whether a compound has the effect to prolong the QT interval in the electrocardiogram in man, wherein the assay is a binding assay using labelled dofetilide binding to the HERG K⁺ channel.

2. The assay of claim 1, wherein the HERG K⁺ channel is human.

3. The assay of claim 2, wherein the ligand is labelled dofetilide.

4. The ligand binding assay of claim 3, wherein the dofetilide is radiolabeled.

5. The ligand binding assay of claim 4, wherein the radiolabel is tritium (³H).

6. The assay of claim 1, wherein the following steps are included:
a) incubation of cells or membranes expressing the HERG K⁺ channel with labelled dofetilide in the presence or absence of different amounts of a test compound or a mixture of test compounds;
b) determination of specifically bound label for each sample;
c) calculation of the inhibition of labeled doetilide binding by the test compound or mixture of test compounds.

7. The assay of claim 6, wherein an additional step is the calculation of the compound concentration that gives 20% inhibition of dofetilide binding to HERG K⁺ channels (IC₂₀), and the comparison of this value with the free compound concentration required for the desired therapeutic effect of the compound.

8. The ligand binding assay of claim 6, wherein bound and unbound labelled dofetilide are separated by filtration.

9. The ligand binding assay as claimed in claim 1, wherein the assay is performed by scintillation proximity assay.
